(19) United States
(12) Patent Application Publication
(10) Pub. No.: US 2002/0010168 A1
(43) Pub. Date: Jan. 24, 2002

(54) USE OF BOSWELLIC ACID AND ITS DERIVATIVES FOR INHIBITING NORMAL AND INCREASED LEUCOCYTIC ELASTASE OR PLASMIN ACTIVITY

(76) Inventors: HERMANN P.T. AMMON, TUBINGEN (DE); HASAN SAFAYHI, TUBINGEN (DE)

Correspondence Address: BURNS DOANE SWECKER & MATHIS L L P, POST OFFICE BOX 1404, ALEXANDRIA, VA, 22313-1404, US

(21) Appl. No.: 09/011,977

(22) PCT Filed: Apr. 22, 1996
(86) PCT No.: PCT/EP96/03705

(30) Foreign Application Priority Data
Aug. 23, 1995 (DE) 195 31 067.5

(51) Int. Cl. A61K 31/19; A01N 37/00; A61K 31/43; A01N 27/00; A61K 31/015

(52) U.S. Cl. 514/198; 514/765

(57) ABSTRACT

The invention concerns the use of pure boswellic acid, a physiologically acceptable salt, a derivative, a salt of the derivative or a plant preparation containing boswellic acid for preventing and/or combatting diseases which are caused by increased leucocytic elastase or plasmin activity or can be treated by the inhibition of normal leucocytic elastase or plasmin activity, in human or veterinary medicine.

The invention further concerns the use of pure boswellic acid or a physiologically acceptable salt, a derivative, a salt of the derivative or a plant preparation containing boswellic acid for preparing a medicament for treating diseases which are caused by increased leucocytic elastase or plasmin activity or can be treated by the inhibition of normal leucocytic elastase or plasmin activity, in human or veterinary medicine.

The invention concerns the use of pure boswellic acid, a physiologically acceptable salt, a derivative, a salt of the derivative or a plant preparation containing boswellic acid for preventing and/or combatting diseases which are caused by increased leucocytic elastase or plasmin activity or can be treated by the inhibition of normal leucocytic elastase or plasmin activity, in human or veterinary medicine.

The invention further concerns the use of pure boswellic acid or a physiologically acceptable salt, a derivative, a salt of the derivative or a plant preparation containing boswellic acid for preparing a medicament for treating diseases which are caused by increased leucocytic elastase or plasmin activity or can be treated by the inhibition of normal leucocytic elastase or plasmin activity, in human or veterinary medicine.

According to the invention, use is made thereof particularly in the case of pulmonary emphysema, acute respiratory distress syndrome, shock lung, cystic fibrosis (mucoviscidosis), chronic bronchitis, glomerulonephritis and rheumatoid arthritis, which are caused by increased leucocytic elastase activity, tumors and neoplasm or tumor metastases, which are caused by increased plasmin activity or can be treated by the inhibition of normal leucocytic elastase or plasmin activity.

EP-A-552 657 discloses the use of boswellic acid for preventing or treating inflammatory diseases accompanied by an increased leucotriene formation. However, this citation does not mention the connection between a leucocytic elastase or plasmin activity and diseases such as cystic fibrosis or chronic bronchitis.

Int. J. Immunopharmacol., 14(7), 1992, pp. 1139-1143, Kapil, A. et al., describes the inhibitory effect of boswellic acid on the complement system, which in the final analysis has a generally anti-inflammatory effect. However, the connection of leucocytic elastase and plasmin activity with concrete symptoms and their successful treatment with boswellic acid is not mentioned herein.

Int. J. Immunopharmacol. 11(6), 1989, pp. 647-652, Sharma, M. L., et al., refers to boswellic acid as promising preparation against arthritis. In this connection, the oral administration of boswellic acids shall cause a reduction of the leucocyte amount. However, no connection is established between leucocytic elastase/plasmin and boswellic acid.

Ann. Rev. Med. 36, 1985, pp. 207-216, Janoff, A., characterizes the part which elastase from neutrophilic granulocytes plays in the degradation of tissue and emphasizes the problem occurring in the case of the uncontrolled release of this enzyme and the accompanying necessity of specific elastase inhibitors. However, the suitability of boswellic acid for solving this problem is not mentioned.

### LEUCOCYTIC ELASTASE

### Occurrence and Biological Activity of Human Leucocytic Elastase

Human leucocytic elastase (HLE; EC 3.4.21.37) is a glycoprotein having protease activity. It is stored in inactivated form in neutrophilic granulocytes (PMNL) of humans, released from granules of these cells upon activation of the neutrophilic granulocytes and, as enzyme (serine protease), it then catalyzes the proteolytic degradation of elastin, collagen, fibronectin and further proteins.

### Pathophysiological Significance

Together with other inflammatory mediators, the serine protease activity of human leucocytic elastase takes part in the formation and maintenance of pathologic changes and the aggravation of such processes—particularly due to the degradation of components of the framework of many organs and tissues. In so far, human leucocytic elastase is attributed to play a part in the following diseases of humans (for a survey: cf. Janoff, Annu. Rev. Med. 36: 207-216, 1985):

pulmonary emphysema, acute respiratory distress syndrome, shock lung,

cystic fibrosis (mucoviscidosis),

chronic bronchitis,

glomerulonephritis,

rheumatoid arthritis.

In general, participation of human leucocytic elastase is postulated in catabolic processes of inflammations of various genesis, which are accompanied by neutrophilic granulocyte infiltration. From basic findings obtained in connection with isolated cells, it can be concluded that it plays a part in the endotoxin-triggered hepatic damage conveyed by conveyed by neutrophilic granulocytes (Sauer et al., Naunyn-S. Arch. Pharmacol. 351 S: Abstract. 495, 1995).

### Inhibitors of Human Leucocytic Elastase

A plurality of naturally occurring and synthetic inhibitors for human leucocytic elastase are known (Groutas 1987, Med. Res. Rev. 7: 227-241; Bode et al. 1989, Biochemistry 28: 1951-1963). The effectiveness of some of these compounds is shown in experimental animal models (Powers 1983, Am. Rev. Respir. Dis. 127: p. 54 - p. 58; Schnebli 1985, in Handbook of Inflammation, Eds: Bonta, Bray & Parnham, Vol. 5: 321-333, Elsevier Sci. Publ., Amsterdam; Soskel et al. 1986, Am. Rev. Respir. Dis. 133: 635638). The inhibition of human leucoytic elastase by some pentacyclic triterpene derivatives was shown. In this connection, the effectiveness of the individual derivatives was different (Ki values 4 to 185 μM). Ursolic acid (Ki=4 to 6 μM) was the most effective one. The boswellic acid series was not tested in this study (Ying et al. 1991, Biochem. J. 277: 521-526).

### Method of Measuring Human Leucocytic Elastase Activity in Vitro

The activity was determined photometrically with pure human leucocytic elastase (Calbiochem) and the substrate MeO-Suc-Ala-Ala-Pro-Val-p-nitroanilide in Dulbecco's phosphate-buffered salt solution (DPBS) in the presence of 10% dimethyl sulfoxide (DMSO) at room temperature and 405 nm (Bieth et al. 1974, Biochem. Meth. 11: 350-357).

### Method of Measuring the Chymotrypsin (CT) Activity in Vitro

The effect which acetyl-11-keto-β-boswellic acid (AKBA) has on the pure chymotrypsin activity (Sigma) was also determined to investigate a possible non-selective inhibition of various serine proteases. The test was carried out photometrically with MeO-Suc-Ala-Ala-Pro-Phe-p-nitroanilide as a substrate in Dulbecco's phosphate-buffered salt solution (DPBS) in the presence of 10% dimethyl sulfoxide at room temperature and 410 nm.

### Results of the Inhibition of Leucocytic Elastase Activity by Boswellic Acids

In an established in vitro test system, the activities of two serine protease enzymes (human leucocytic elastase and chymotrypsin) were measured. Pure enzymes from Calbiochem and SIGMA, respectively, were used, and the enzyme activity was determined as the rate at which the enzymes released p-nitroaniline from their substrates by hydrolysis per time unit. Three series of measurement were made for determining the HLE activity at three different substrate concentrations. The 100% controls did not contain any test substances but only the corresponding amount of solvent. The effects which differing concentrations of acetyl-11-keto-β-boswellic acid (AKBA) had on the human leucocytic elastase in these three series of measurement are shown as percentage of 100% controls as mean values ±S.D. of three independent measurements each.

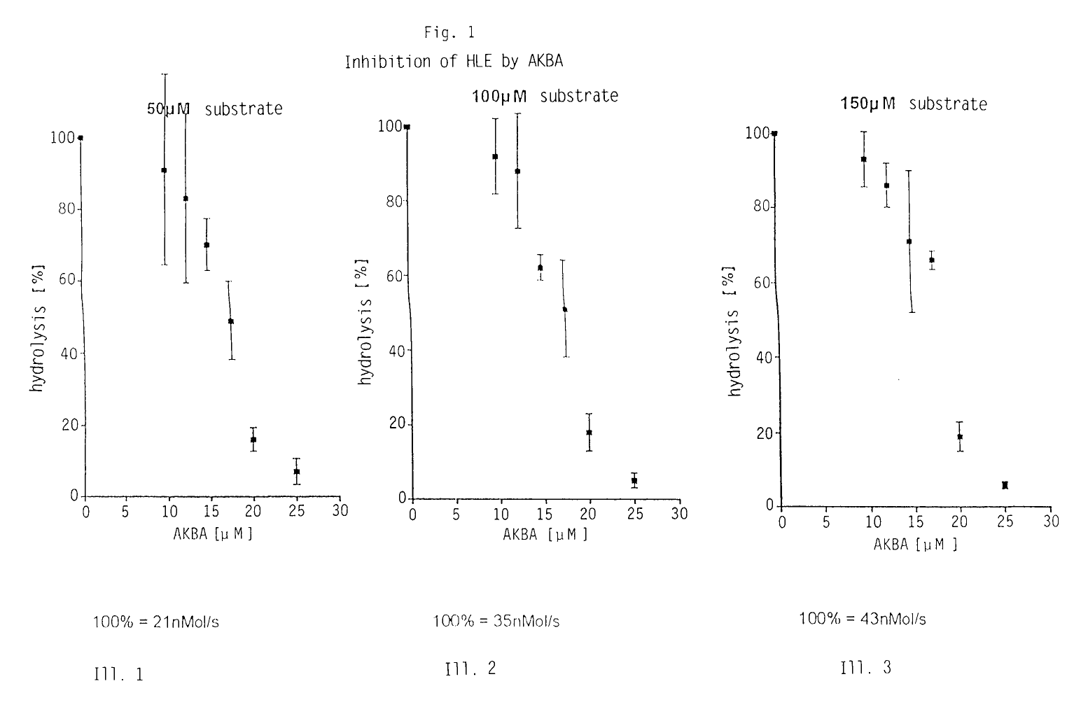

1. Acetyl-11-keto-β-boswellic acid inhibited human leucocytic elastase in a concentration-depending manner with IC50 values of about 17 μM (at substrate concentrations of 50, 100 and 150 μM). In the case of 20 μM of acetyl-11-keto-β-boswellic acid, a residual activity of 18% remained; in the case of 20 μM of ursolic acid, the residual activity was 11% (cf. FIG. 1).

2. At a concentration of 20 μM each in the test, the following compounds used as reference substances had no inhibitory effect in this system: as pentacyclic triterpene derivative: glycerrhitinic acid, as reducing and/or competitive 5-lipoxygenase inhibitors: NDGA, MK886 and ICI230,487, as steroids: cortisol and testosterone, and as polyunsaturated long-chain fatty acid: arachidonic acid.

3. Acetyl-11-keto-β-boswellic acid (AKBA) had no significant inhibitory effect on the chymotrypsin activity at concentrations of 50 and 100 μM (89 and 85 of the control activity), whereas ursolic acid inhibited the activity of chymotrypsin at 50 μM to 56% and at 100 μM to 30% of the control activity of chymotrypsin.

### Summary of the Results Concerning the Inhibition of Human Leucocytic Elastase

It follows from these results that acetyl-1l-keto-β-boswellic acid also has an inhibitory effect on human leucoytic elastase in addition to its 5-lipoxygenase-inhibiting property and the accompanying reduction of leucotriene biosynthesis.

The inhibition of this enzyme is of importance because during the pathophysiological processes of the above-mentioned diseases (pulmonary emphysema, acute respiratory distress syndrome, shock lung, chronic bronchitis, cystic fibrosis, glomerulonephritis and rheumatoid arthritis) the human leucocytic elastase released from the activated neutrophilic granulocytes plays an important part in the destruction of functional tissue and thus is responsible for the damage caused by these diseases in the lungs, kidneys and joints. Therefore, it should be possible to prevent the damage of the organs resulting from these diseases by the inhibition of human leucocytic elastase by boswellic acid or the derivatives thereof. Selective inhibitors of human leucocytic elastase have not been available up to the present. However, non-selective inhibitors are not suitable for pharmacotherapy, since they can cause serious undesired effects because they inhibit other proteases as well. Moreover, boswellic acids and derivatives are well resorbed and, as has been proved, not toxic. Another advantage occurring when boswellic acids are used consists in that the synchronous inhibition of two inflammation-promoting mediator systems of leucocytes could be utilized synergistically by this monosubstance for the pharmacotherapy of a number of diseases which can presently be controlled only insufficiently. Although other pentacyclic triterpenes can also inhibit HLE (e.g. ursolic acid), other pentacyclic triterpenes—with the exception of boswellic acids—have no effect on the leucotriene biosynthesis (Safayhi et al. 1995, Mol. Pharmacol. in press). As in the class of dioxygenases for 5-lipoxygenase (Safayhi et al. JPET 1992), AKBA also has a certain inhibitory effect on HLE among the serine proteases, as shown by the lacking effect on chymotrypsin, a digestive enzyme from the serine protease class.

### PLASMIN

### Occurrence and Biological Activity

Like human leucocytic elastase, plasmin (EC 3.4.21.7.) is a serine protease. As an enzyme plasmin catalyzes the hydrolysis of peptide bonds, in which arginine and lysine take part, and thus the degradation of a number of proteins and peptides. Plasmin occurs in the blood as the inactive precursor plasminogen, and is formed by proteolytic activation from the precursor (plasminogen). The increased activity of plasmin is held responsible for the destruction of cell framework proteins occurring in the course of many diseases, but also for the invasive growth and metastatic spread of malignant tumors, which is accompanied by the destruction of endogenous functional tissue (Wangh et al., Int. J. Cancer. 1994, 58: 650-657). Moreover, plasmin also activates what is called growth factors which can also stimulate the reproduction of tumors (Campbell et al., J. Cell. Physiol. 1994, 159: 1-10). Therefore, it appears to be possible to inhibit the growth and metastatic spread of many kinds of cancer by inhibition of the plasmin activity.

### Inhibition of the Plasmin Activity by Boswellic Acids

### Method: Measurement of the Plasmin Activity

The activity of human plasmin (SIGMA) was determined photometrically in vitro with the substrate D-Ile-Phe-Lys-pNA (Cs-Szabo et al., Thrombosis Res. 1980, 20: 199-206). The plasmin activity is referred to as the release of p-nitronaniline per minute (nmole/min) from the substrate as mean values ±S.D. of n=3 measurements each.

### Results

The pentacyclic triterpenic acids from the boswellic acid series, β-boswellic acid (β-BA) and acetyl-11-keto-β-boswellic acid (AKBA), inhibit the plasmin activity with comparable effectiveness with half-maximum inhibition constants of about 4 to 6 μM (FIG. 2, Ills. 1 and 2). In contrast to the inhibition of HLE (human leucocytic elastase) , ursolic acid is markedly less effective with respect to the effect on the plasmin activity (IC50 of about 15 μM; FIG. 2, Ill. 3), whereas amyrin does not influence significantly the plasmin activity at concentrations up to 50 μM (not shown).

### Summary

In an in vitro test system, β-BA and AKBA inhibited almost completely the plasmin activity at concentrations which following oral administration of olibanum or frankincense extracts can be reached in the blood of humans. Other pentacyclic triterpenic acids are either substantially weaker (ursolic acid) or not effective at all (amyrin) in this system.

Since the plasmin activity represents one of the essential mechanisms of malignant tumor growth, which is accompanied by the destruction of functional tissue in the host organism, it appears to be likely that the formation of carcinomas and sarcomas could be prevented by the use of boswellic acids.

Up to the present, no satisfactory method of treatment is available in the therapy of diseases such as pulmonary emphysema, acute respiratory distress syndrome, shock lung, cystic fibrosis (mucoviscidosis), chronic bronchitis, glomerulonephritis and rheumatoid arthritis, diseases which are caused by increased leucocytic elastase activity, and tumors and neoplasm or tumor metastases which are caused by increased plasmin activity.

Chronic bronchitis, pulmonary emphysema, acute respiratory distress syndrome and shock lung (ARDS) belong to the diseases of the respiratory apparatus which may be due to various causes. Even though differently defined disease entities are concerned, they overlap considerably as regards pathophysiological processes, diagnostic measures and therapeutic approaches. While in the early stage a fundamental improvement can be achieved by eliminating the noxious substances (e.g. ban on smoking, change of job, antibiotics . . . ), usually only an improvement of the symptoms is possible (improvement of the secretory drainage, oxygen respiration) when the disease proceeds, which is accompanied by the destruction of the functional tissue.

In the case of an emphysema recombinant (α1-antitrypsin can be substituted when an α1-antitrypsin deficiency (deficiency of endogenous protease inhibitor) is given. In the case of ARDS: Acute/Adult Respiratory Distress Syndrome (shock lung), an acutely life-threatening pulmonary dysfunction which proceeds like a shock and represents the most frequent cause of death in patients who survived the early stage of a shock resulting from a primarily non-pulmonary cause and is a dangerous complication occurring in traumatized and operated patients, aprotinin, corticoids, heparin and low-molecular dextrans are used as medicaments. Depending on the duration of respiratory insufficiency, lethality is still 30 to 90% these days.

Cystic fibrosis (mucoviscidosis) is a hereditary metabolic anomaly which due to a generalized dysfunction of exocrine glands (increased production and high viscosity of the secretion of the mucous glands of the bronchi and the digestive apparatus) leads to serious complications in the region of the respiratory apparatus and in the pancreas, which progressively result in the degradation of functional tissue in the affected organs. The treatment focuses predominantly on the prevention of complications by the continuous care of the respiratory apparatus (physiotherapy, inhalations, early and well-calculated antibiosis).

In the case of arthritis (inflammations of a joint), processes proceeding together with hydrolizing enzymes (e.g. leucocytic elastase) result in degenerative changes of the capsule. By way of medicament, the secondary pain or after-pain is prevented symptomatically using non-steroidal antirheumatics. Treatment of the causally responsible secondary damage occurring in connection with primary-inflammatory arthritis is possible by means of steroids (corticoids) but out of the question because of the serious undesired side-effects. A direct inhibition of catabolic processes proceeding along with leucocytic elastase is not possible these days. Glomerulonephritis is a general term for widely differing renal diseases accompanied by inflammatory processes which may result in degenerative processes including terminal renal failure. Causal treatment is lacking. In the case of some forms, corticoids may help, serious undesired effect having to be accepted.

For treating the above-mentioned diseases, the pharmaceutical industry is hectically searching for leucocytic elastase and plasmin inhibitors which are non-toxic.

It is the object of the present invention to provide the use of preparations which serve for preventing and/or combatting diseases which are accompanied by an excessive leucocytic elastase or plasmin activity or can be treated by the inhibition of normal leucocytic elastase or plasmin activity. The preparations provided according to the invention shall be particularly capable of curing the above-mentioned diseases. It shall be possible to administer the medicaments used according to the invention for a long period of time, without any side-effects occurring. The medicament used according to the invention shall be non-toxic and well tolerated by the patients.

Surprisingly it has now been found that boswellic acid, a physiologically acceptable salt, a derivative, a salt of the derivative or a plant preparation containing boswellic acid can be used for preventing and/or combatting diseases which are caused by increased leucocytic elastase or plasmin activity, in human or veterinary medicine.

The plant Boswellia serrata and other Boswellia kinds contain ingredients which are capable of selectively inhibiting the leucocytic elastase formation and the leucocytic plasmin activity. According to the statements made by Pardhy & Bhattacharyya (Ind. J. Chem., 16B: 176-178, 1978) Boswellia serrata contains substantially the following ingredients: β-boswellic acid, acetyl-β-boswellic acid, acetyl-11-keto-β-boswellic acid, 11-keto-β-boswellic acid. However no pharmacological investigations have been made thereon so far in the field of human leucocytic elastase inhibition or leucocytic plasmin inhibition.

The structural formulae of boswellic acid and some of their derivatives are listed below:

(A):

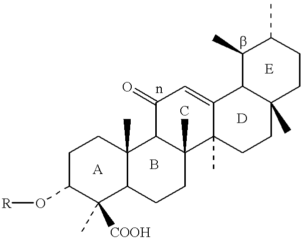

R=H: 11-keto-β-boswellic acid

R=acetyl: acetyl-11-keto-β-boswellic acid

R=formyl: formyl-11-keto-β-boswellic acid

(B):

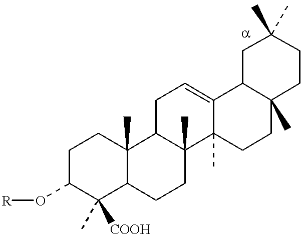

R=H: α-boswellic acid

R=acetyl: acetyl-α-boswellic acid

R=formyl: formyl-α-boswellic acid

β-boswellic acid is preferably used as boswellic acid. According to the literature, it is isolated from Boswellia serrata or other known plants containing boswellic acid. β-boswellic acid may contain minor amounts of α- or γ-boswellic acid. Sodium, potassium, ammonium, calcium salts can be used as physiologically acceptable salts of boswellic acid. Lower alkyl esters obtained by esterification of the carboxyl group with a C₁-C₆ alcohol, preferably methyl ester, or esters obtained by esterification of the hydroxyl group with a physiologically compatible carboxylic acid, are used as derivatives of boswellic acid. β-boswellic acid acetate, β-boswellic acid formate, β-boswellic acid methyl ester, acetyl-β-boswellic acid, acetyl-11-keto-β-boswellic acid and 11-keto-β-boswellic acid are preferred derivatives.

According to the invention it is also possible to use a plant preparation containing boswellic acid. According to the invention preparations which are obtained from the resin are used. Olibanum and olibanum extract are especially preferred.

An especially preferred plant preparation containing boswellic acid is phytopharmacon H 15 which is sold by the company of Ayurmedica, Pöcking, Germany. It is a lipophilic extract from Boswellia serrata. This medicament available on prescription only contains a dry extract from olibanum as active substance. The commercial products tablet and granulate are composed as follows:

1 tablet contains 400 mg of dry extract from olibanum (4.2-5.9:1), extracting agent: chloroform/methanol

1 g of granulate contains 500 mg of dry extract from olibanum (4.2-5.9:1), extracting agent: chloroform/methanol.

According to the invention it is possible to use natural, synthetic compounds and the mixtures thereof.

According to the invention it is also possible to use them together with other chemical pharmaceutical substances and/or other plant medicaments.

Boswellic acid is administered according to the invention as required. Since it shows little toxicity, its dosage is not critical and can easily be varied by the physician depending on the severity of the disease, the weight of the patient to be treated and the duration of treatment.

Unit doses may be administered one to four times daily, for example. The accurate dose depends on the way of administration, the condition to be treated, the patient's weight, etc. By nature, it may be required to vary the dose as a matter of routine, depending on the age and weight of the patient as well as the severity of the condition to be treated.

The preparations used according to the invention can be formulated in known manner by using one or more pharmaceutically acceptable carriers or diluents. The preparations can be formulated for intraperitoneal, oral, buccal, parenteral, rectal, intramuscular, topical, subcutaneous, intraarticular, intravenous or intranasal administration or in a way suitable for administration by inhalation or insufflation. Preparations of the compounds for oral administration are preferred.

The pharmaceutical preparations can be made in the form of tablets, dragees, capsules, solutions, emulsions, ointments, creams, inhalants, aerosols or suppositories.

The pharmaceutical preparations for oral administration may be available in the form of tablets or capsules, for example, which are produced according to methods known per se with pharmaceutically acceptable diluents, such as binders (pregelatinized corn starch, polyvinylpyrrolidone or hydroxypropyl methyl cellulose, for example), fillers (e.g. lactose, saccharose, mannitol, corn starch, microcrystalline cellulose or calcium hydrogen phosphate); lubricants (e.g. stearic acid, polyethylene glycol, magnesium stearate, talcum or silicon dioxide); disintegrating agents (e.g. potato starch, sodium starch glycolate or sodium carboxymethyl cellulose); or wetting agents (e.g. sodium lauryl sulfate). The tablets can be coated according to methods known per se. Liquid preparations for oral administration may be available in the form of e.g. aqueous or oily solutions, syrups, elixirs, emulsions or suspensions, or they may be available as dry product for the constitution with water or another suitable carrier prior to use. Such liquid preparations can be produced according to methods known per se with pharmaceutically acceptable additives, such as suspending agents (e.g. sorbitol syrup, cellulose derivatives, glucose/sugar syrup, gelatin, aluminum stearate gel or hydrogenated edible fats); emulsifiers (for example, lecithin, gum arabic or sorbitan monooleate); non-aqueous carriers (e.g. almond oil, oily esters, ethyl alcohol or fractional plant oils); and preservatives (methyl or propyl-p-hydroxy-benzoates or sorbic acid, for example). The liquid preparations may also contain generally known buffers, flavoring agents, colorants and sweeteners, as required.

For the parenteral administration the compounds can be formulated for injection, preferably intravenous, intramuscular or subcutaneous injection. Preparations for injection may be available in the form of single doses, e.g. in ampoules, or multiple-dose containers with a preservative added. The preparations can be available in the form of suspensions, solutions or emulsions in oily or aqueous carriers and contain preparation aids, such as suspending agents, stabilizers and/or dispersants, and/or agents for adjusting the tonicity of the solution. As an alternative, the active ingredient may be available in the form of a powder for the constitution with a suitable carrier, e.g. sterile pyrogen-free water, prior to use.

The compounds may also be formulated as rectal preparations such as suppositories, e.g. those which contain generally known base materials for suppositories, such as cocoa butter or other glycerides.

For intranasal administration, the compounds can be used as liquid sprays, in the form of drops or as snuff powder.

For administration by inhalation, the compounds are usefully supplied in the form of an aerosol spray from a pressurized pack by using suitable propellants or from an atomizer. In the case of a pressurized aerosol, the unit dose is determined by providing a valve which releases a metered amount. Capsules and cartridges made e.g. of gelatin for use in an inhalator or an insufflator can be prepared such that they contain a powder mixture consisting of a compound used according to the invention and a suitable basic powder material such as lactose or starch.

The following examples explain the use according to the invention.

### EXAMPLE 1

### Tablets for Oral Administration

A. Direct compression

| | | |
|---|---|---|
| (1) | | |
| active substance: boswellic acid | 15-30 | mg/tablet |
| (and powderized drug, respectively | 0.5-1.0 | g/tablet) |
| magnesium stearate BP | 0.65 | mg/tablet |
| anhydrous lactose | 80 | mg/tablet |

The active substance is mixed with anhydrous lactose and the magnesium stearate, and the mixture is sieved. The resulting mixture is compressed into tablets by means of a tabletting machine.

| | | |
|---|---|---|
| (2) | | |
| Active substance: boswellic acid | 15-30 | mg/tablet |
| (and powderized drug, respectively | 0.5-1.0 | g/tablet) |
| magnesium stearate BP | 0.7 | mg/tablet |
| microcrystalline cellulose NF | 100 | mg/tablet |

The active substance is sieved and mixed with the microcrystalline cellulose and magnesium stearate. The resulting mixture is compressed into tablets by means of a tabletting machine.

B. Wet granulation

| | | |
|---|---|---|
| Active substance: boswellic acid | 15-30 | mg/tablet |
| (and powderized drug, respectively | 0.5-1.0 | g/tablet) |
| lactose BP | 150.0 | mg/tablet |
| starch BP | 30.0 | mg/tablet |
| pregelatinized corn starch BP | 15.0 | mg/tablet |
| magnesium stearate BP | 1.5 | mg/tablet |

The active substance is sieved through a suitable screen and mixed with the lactose, starch and pregelatinized corn starch. Suitable volumes of purified water are added, and the powder is granulated. After drying, the granulate is sieved and mixed with the magnesium stearate. The granulate is then compressed into tablets by means of punches having a suitable diameter.

Tablets of differing composition can be produced by changing the ratio of active substance to lactose or the compression weight and using corresponding punches.

### EXAMPLE 2

Capsules

| | | |
|---|---|---|
| Active substance: boswellic acid | 15-30 | mg/capsule |
| (and granulated drug, respectively | 0.5-1.0 | g/capsule) |
| free-flowing starch | 150.00 | mg/capsule |
| magnesium stearate BP | 1.00 | mg/capsule |

The active substance is sieved and mixed with other components. The mixture is filled into hard gelatin capsules No. 2 by using a suitable apparatus. Other capsules can be produced by changing the input weight and, if necessary, by changing the capsule size correspondingly.

### EXAMPLE 3

Syrup

| saccharose-free preparation | | mg/5 ml dose |
|---|---|---|
| active substance: boswellic acid | | 15-30 |
| hydroxypropyl methyl cellulose USP | | 22.5 |
| (viscosity type 4000) | | |
| buffer | | |
| flavoring agent | | |
| coloring matter | {close oversize parenthesis} | as required |
| preservative | | |
| sweetener | | |
| purified water to | | 5.0 ml |

The hydroxypropyl methyl cellulose is dispersed in hot water, cooled down and then mixed with an aqueous suspension containing the active substance and the other components of the preparation. The resulting solution is adjusted to its volume and mixed.

### EXAMPLE 4

| Suspension | | mg/5 ml dose |
|---|---|---|
| active substance: boswellic acid | | 15-30 |
| (and powderized drug, respectively | | 0.5-1.0 g) |
| (dried drug extract correspondingly) | | |
| aluminum monostearate | | 75.00 |
| sweetener | | |
| flavoring agent | {close oversize parenthesis} | as required |
| coloring matter | | |
| fractional coconut oil to | | 5.00 |

The aluminum monostearate is dispersed in about 90% of the fractional coconut oil. The resulting suspension is heated to 115° C. by stirring and then cooled down. The sweeteners, flavoring agents and coloring matters are added, and the active substance is dispersed. The suspension is adjusted with the rest of the fractional coconut oil to the volume and mixed.

### EXAMPLE 5

Sublingual tablet

| | | |
|---|---|---|
| Active substance: boswellic acid | 15-30 | mg/tablet |
| (and drug extract, respectively | 0.5-1.0 | g/tablet) |
| moldable sugar NF | 50.5 | mg/tablet |
| magnesium stearate BP | 0.5 | mg/tablet |

The active substance is sieved through a suitable screen, mixed with the other components and compressed by means of suitable punches. Tablets of differing strength can be produced by changing the ratio of active substance to carrier or the compression weight.

### EXAMPLE 6

Suppositories for rectal administration

| | | |
|---|---|---|
| Active substance: boswellic acid | 15-30 | mg |
| Witepsol H15⁺ to | 1.0 | g |

⁺suitable quality of Adeps solidus Ph.Eur.

A suspension of the active substance in molten Witepsol is produced and filled into 1-g suppository molds by means of a suitable device.

### EXAMPLE 7

Injection for intravenous administration

| | | |
|---|---|---|
| Active substance: boswellic acid | 15-30 | mg/ml |
| sodium chloride-intravenous | | |
| infusion BP | | |
| 0.9% wt./vol. to | 1 | ml |
| batch size | 2500 | ml |

The active substance is dissolved in part of the sodium chloride-intravenous infusion, the solution is adjusted with the sodium chloride-intravenous infusion to the volume, and the solution is thoroughly mixed. The solution is filled into clear, type 1, 10-ml glass ampoules and sealed in nitrogen in the head space by melting off the glass. The ampoules are sterilized by heating in an autoclave at 120° C. for not less than 20 minutes.

### EXAMPLE 8

Cartridge for inhalation

| | | |
|---|---|---|
| Active substance (micronized): | 15-30 | mg/cartridge |
| boswellic acid | | |
| lactose BP | 25.00 | |

The active substance is micronized in a jet mill to give a fine particle size range and then mixed with the lactose. The powder mixture is filled into hard gelatin capsules No. 3.

### EXAMPLE 9

| | |
|---|---|
| Nasal spray | |
| Active substance: boswellic acid | 1.5-3.0%/vol. |
| preservative ) | as required |
| sodium chloride BP ) | |
| purified water BP to | 100 |
| supply weight | 100 mg (equivalent |
| | to 7 mg active |
| | substance) |

The active substance, preservative and sodium chloride are dissolved in part of the water. The solution is adjusted with water to the volume, and the solution is thoroughly mixed.

1. Use of pure boswellic acid, a physiologically acceptable salt, a derivative, a salt of the derivative or a plant preparation containing boswellic acid for preventing and/or combatting diseases which are caused by increased leucocytic elastase or plasmin activity or can be treated by the inhibition of normal leucocytic elastase or plasmin activity, in human or veterinary medicine.

2. Use according to claim 1, characterized in that use is made in the case of pulmonary emphysema, acute respiratory distress syndrome, shock lung, cystic fibrosis (mucoviscidosis), chronic bronchitis, glomerulonephritis and rheumatoid arthritis, which are caused by increased leucocytic elastase activity, and in the case of tumors and neoplasm or tumor metastases which are caused by increased plasmin activity.

3. Use according to claim 1 or 2, characterized in that use is made intraperitoneally, orally, buccally, rectally, intramuscularly, topically, subcutaneously, intraarticularly, intravenously or inhalationally.

4. Use according to at least one of claims 1 to 3, characterized in that use is made in the form of tablets, dragees, capsules, solutions, emulsions, ointments, creams, inhalants, aerosols or suppositories.

5. Use of pure boswellic acid or a physiologically acceptable salt, a derivative, a salt of the derivative or a plant preparation containing boswellic acid for the preparation of a medicament for treating diseases which are caused by increased leucocytic elastase or plasmin activity or which can be treated by the inhibition of normal leucocytic elastase or plasmin activity, in human or veterinary medicine.

6. Use according to claim 5, characterized in that a medicament is produced for the treatment of pulmonary emphysema, acute respiratory distress syndrome, shock lung, cystic fibrosis (mucoviscidosis), chronic bronchitis, glomerulonephritis and rheumatoid arthritis, which are caused by increased leucocytic elastase activity, and in the case of tumors and neoplasm or tumor metastases which are caused by increased plasmin activity.

7. Use according to claim 6, characterized in that use is made for the preparation of a medicament for the intraperitoneal, oral, buccal, rectal, intramuscular, topical, subcutaneous, intraarticular, intravenous or inhalational administration.

8. Use according to claim 6 or 7, characterized in that use is made for the preparation of a medicament in the form of tablets, dragees, capsules, solutions, emulsions, ointments, creams, inhalants, aerosols or suppositories.

9. Use according to at least one of claims 1 to 8, characterized in that use is made together with other chemically pure medicinal substances, and/or plant medicaments.
